(19) United States
(12) Patent Application Publication
(10) Pub. No.: US 2007/0184099 A1
(43) Pub. Date: Aug. 9, 2007

(54) Films for use as dosage forms

(75) Inventor: Edward Zbygniew Nowak, Cambridge (GB)

Correspondence Address: MCDERMOTT WILL & EMERY LLP, 18191 VON KARMAN AVE., SUITE 500, IRVINE, CA, 92612-7108, US

(73) Assignee: BIOPROGRESS TECHNOLOGY LIMITED, CAMBRIDGE (GB)

(21) Appl. No.: 10/590,038

(22) PCT Filed: Feb. 18, 2005
(86) PCT No.: PCT/GB05/00615

§ 371 Date: Nov. 17, 2006

(30) Foreign Application Priority Data
Feb. 20, 2004 (GB) 0403808.9

(51) Int. Cl. A61K 9/48; A61K 9/24

(52) U.S. Cl. 424/451; 424/473; 424/472

(57) ABSTRACT

Non gelatin film materials e.g. films of modified cellulose materials find use as dosage forms. Substances are incorporated into the film matrix and films thus prepared may be administered really, or otherwise internally, or epidermally. The administable form may comprise a matrix which contains at least one water soluble polymer in the form of a film, in addition to at least one active ingredient, to produce a therapeutic, organoleptic or cosmetic effect.

### FIELD OF INVENTION

This invention relates to non gelatin film materials, for example, films of modified cellulose materials (or cellulose derivatives), and the incorporation of one or more active ingredients.

This invention further relates to film products and methods of preparation thereof and includes associated processes for the incorporation of substances into a film matrix.

Films thus prepared may be administered orally or otherwise internally or epidermally, or indeed in any manner where it can release one or more active ingredients either rapidly or at a controlled rate.

The administrable form may comprise a matrix which contains at least one water-soluble polymer in the form of a film; in addition at to least one active ingredient to produce a therapeutic, organoleptic or cosmetic effect.

### BACKGROUND OF THE INVENTION

As an alternative to tablets and pills, films may be used to carry active ingredients such as drugs, pharmaceuticals, and the like. However, historically films and the process of making drug delivery systems therefrom have suffered from a number of unfavorable characteristics that have not allowed them to be used in practice.

Water-soluble films cast from aqueous solutions containing medications can suffer from the aggregation or conglomeration of particles. Self-aggregation of any active ingredient will make the film inherently non-uniform in its composition. If such films were to include low dosages of an active ingredient, it is possible that portions of the film may be substantially devoid of any e.g. medication.

Furthermore, conventional film casting employs the use the time-consuming drying equipment such as a high-temperature air-bath, drying ovens, drying tunnels, vacuum driers, or other such drying equipment. The long length of drying time aids in promoting the aggregation of the active ingredient and/or other adjuvant. Such process also run the risk of exposing the active ingredient, i.e., a drug or vitamin or other components to prolonged exposure to moisture and elevated temperatures, which may render it ineffective or even harmful.

In an example, where the film is hot melt extruded, as in the case with HPC, then it could be difficult to mix an active ingredient into the film without degrading the active ingredient in some way.

Other factors, such as mixing techniques also play a role in the manufacture of films containing active ingredients or pharmaceuticals. During film solution preparation air is often trapped in the solution and needs to be removed. This can result in the separation of actives which are suspended in the solution (a process commonly known as Froth Floatation) which in this instance would be undesirable.

Additionally if trapped air is not removed then voids are produced in the film during the drying stage. The result is non-uniformity in the final film product.

An alternative to casting film solutions containing active ingredients is to surface coat the active ingredient onto a film substrate. This can result in a hetrogenous system where the active is poorly associated with the film surface resulting in an oily or powdery surface layer prone to abrasion and simply being wiped off during conversion or handling.

### SUMMARY OF THE INVENTION

One object of the present invention relates to production of films containing active ingredients using novel processes.

A active ingredient can be conveniently transported through the surface of a film via a liquid formulation applied on one or more surfaces of the film.

In accordance with one aspect of the present invention, by way of example only, a active ingredient may be dissolved in a hydrophilic, organic system to form a homogeneous solution or dispersion. This solution or dispersion can be then applied to one or more surfaces of a non gelatin polymeric film, e.g. a dry cellulose ether film, resulting in the active ingredient and/or liquid carrier phase being transported through the surface of the ‘dry’ film resulting in a new film composition.

This new film composition may or may not contain all the components of the film and solution. It may have the active ingredient absorbed to a varying degree in the film substrate, for example, the active ingredient may be absorbed evenly within the film substrate or it may be absorbed only near the surface of the film substrate. Variation between these 2 physical states are envisaged. Patterns or bands in the films are contemplated.

The film substrate may remain completely intact or relatively physically unchanged immediately following the incorporation process, and can be converted to any size or shape of unit dosage form. Alternatively, the film substrate may liquefy or dissolve partly or fully, during the incorporation process, but nevertherless finally forming a single discrete film, after curing.

Films according to the present invention are typically made up of one or more soluble polymers or polymers which will otherwise degrade e.g. at the intended site of release of the active ingredient, e.g. in the mouth. Non readily soluble films are also contemplated, as there are situations where this could also be a possible advantage, for example in the controlled release of a medicament.

### DETAILED DESCRIPTION OF THE INVENTION

One aspect of the invention relates to non gelatin films and in particular films made from cellulose ethers.

More particularly, the way in which such films incorporating one or more active ingredients can be produced is herein described.

Such films are useful for delivering a variety of agents to humans and other animals to produce a therapeutic, organoleptic or cosmetic effect.

Selective deposition of active ingredients about or within dosage forms according to the present invention may result in superior storage qualities (e.g. no exposed active) of a dosage form or superior active ingredient release characteristics (favourable zonal depositiona of actives. The selective/accurate deposition of actives may also result in less wastage of active ingredient, during the manufacture of the dosage form. This may also result in less wastage of other materials such as film forming polymers etc.

Typically, cellulose ether films can be prepared by casting an aqueous solution of the cellulose ether onto a heated plate which drives off the water and other fugitive solvents to leave a solid thin film.

Suitable cellulose ethers include hydroxypropyl methylcellulose (HPMC), hydroxy propyl cellulose (HPC), Hydroxy ethyl methyl cellulose (HEMC), Hydroxy ethyl cellulose (HEC), methyl cellulose (MC), carboxy methylcellulose (CMC) (including sodium carboxy methylcellulose) and salts and derivatives of all aforesaid.

Enteric materials that also may be suitable include cellulose Acetate phthalate (CAP), Hydroxy Propyl methyl cellulose phthalate (BPMC-P), Hydroxy propyl methyl cellulose acetate succinate (BPMC-AC), and also, Ethyl Cellulose (EC), Carboxymethyl hydroxyethyl cellulose (CMHEC), and sodium salt of above (Na-CMHEC) (the Na salt would not be regarded as enteric)

The invention is not limited to utilization of cellulose ethers for film formation nor to a film for use in connection with only treating animals or humans, but is intended to utilize any suitable non gelatin film, made in accordance with the method of the present invention, which can release an active ingredient.

It is contemplated that such films however may have particular application in the treatment of animals and humans and perhaps more particularly to the production and use of a film that is suited to ingestion or application otherwise to a human or other animal.

In one preferred embodiment of the invention, a film produced by the method in accordance with the present invention is provided, such film containing a active ingredient suitable for human or animal ingestion.

In an aspect of the invention there is provided a film that is an effective and convenient topical or intra-cavity drug delivery system for applying and delivering controlled dosages of therapeutic agents e.g. onto or into skin or the body.

Controlled drug delivery via the skin, (e.g. in skin care or cosmetics), gynaecological, vaginal, cranial, abdominal, otic, uterine, nasal, sinus, rectal, buccal, oral, ophthalmic, and wound care applications can also be achieved by the use of the product according to the present invention.

The film can be utilized for the delivery of a wide range of pharmaceutically active ingredients. Some therapeutic agents exhibit absoption problems due to solubility, degradation (e.g. in the gastro-intestinal tract), or reduction by extensive metabolism.

Without limiting the invention, examples of therapeutic agents include hypnotics, sedatives, anti-epileptics, awakening agents, psychoneurotropic agents, neuromuscular blocking agents, antispasmodic agents, antihistaminics, atiallergenics, cardiotonics, antiarrhymics, diuretics, hypotensives, vasopressors, antitussive expectorants, thyroid hormones, sexual hormones, antidiabetics, anti tumor agents, antibiotics and chemotherapeutics and narcotics.

Cosmetically active compounds may include breath freshening agents like menthol, or other flavours of fragrances used for oral hygiene and or actives used for dental and/or oral cleansing like quaternary or ammonium bases. The effect of flavours may be enhanced using flavour enhancers like tartaric acid, citric acid, vanillin, and the like.

A particularly suitable cellulose ether is HPMC. To facilitate processing of the films and to increase the apparent flexibility, at least one plasticizer maybe added, such as an edible plasticiser for an oral film. Plasticisers commonly used are polyols, glycols, acetins, carboxylic acids and the esters of these acids, for example polyethylene glycol, glycerin, triacetin, citric acid and triethylcitrate respectively. The plasticisers maybe used individually or in combination and maybe present in any desired amount, particularly from 0 to about 40 percent of the solid film and more particularly from 0 to 20 percent.

Optional ingredients may be added including, without limitation, colourants, emulsifiers, humectants, defoamers and anti block agents. Such optional components are typically added in minor amounts, to aid the processing of the film and typically are less than 10% total by weight based upon the weight of the cellulose ether component.

The base films may be made by a variety of processes, for example by dissolving or dispersing the film components in water or other solvents and drying into film form. Alternatively the film resins could be hot-melt extruded. Additionally a dispersion or solution maybe directly coated or sprayed onto another edible product, such as a tablet or foodstuff and dried to form an edible film. The preferred technique is to have film solution cast and dried to produce a sheet of flexible, thin film.

The base of a film so produced can be seen to act as a ‘building block’ from which the final film product is produced (product film). The ‘building block’ film may be considered as a part-formed film, and the product film may be considered as a homogenous film or a film formed from one or more bands, at least one band being derived from the building block. The ‘building block’ film according to the present invention may be considered as only a part-formed film because further mass is added later as an inevitable result of the method according to the present invention,

A uniform, flat film is found to be suitable to serve as a ‘building block’ film. Such films can be used to form tablets or monoliths, which may comprise many such films.

The next stage in the process according to the present invention is the application of a fluid, e.g. a liquid, to the film. The liquid may be applied by many methods including ink-jet type application. In one embodiment, the ink-jet type apparatus may be modified to apply active(s) and a film forming polymer or polymers and in this way, accurate patterns of doses in the dosage form can be realized.

According to an aspect of the invention, by way of example, application of a solution, suspension or micro-emulsion containing the active ingredient (hereinafter referred to as liquid), onto to one or more surfaces of the ‘building block’ film to produces a new film or product film.

In one aspect of the invention the product film may serve as a dosage form in its own right

In another aspect of the invention the product film may be used to form other dosage forms, e.g. by forming a tablet or monolith composed of many layers of the product film. The product films used may be the same or different, and any range of a combination of different product films. The films may simply be bonded together forming laminate dosage forms of many discrete layers or the product films may be fused or welded together, forming a single mass of material, albeit perhaps with regional zones with varying properties, such as zones with different drugs or drugs in higher concentrations or zones of polymers of differing strengths or solubilities. In one process for producing a tablet or monolith, the product film(s) may not be allowed to be fully cured, and, for example, the liquid deposited about the building block film, is not let to ‘set’ or go into solid form, and another building block film is (immediately) applied to form a ‘sandwich’, and the liquid can/may be taken up by both building block films (perhaps taking up active also), e.g. to form a tri-layer (fused) film. This can process can be repeated many times to form multilayer films, e.g. which may be fused. The process also has the advantage that there is no need for any additional bonding agent, glue or other process to attach the films together, as the liquid used to form the product film has also performed this function of bonding. This process has applications in conveniently producing robust multilayer dosage forms such as monoliths or tablets.

In an aspect of the present invention, complete homogeneity is achieved in the product film.

In another aspect of the present invention, a product film having variations in physical quality and/or chemical composition is produced, e.g. the active may be preferentially distributed in a favourable manner within the film.

The result achieved depends on the chemicals and conditions used in the process according to the present invention.

In an example, one or more surfaces of a ‘dry’ cellulose ether film are coated.

In one embodiment of the invention, for e.g. ease of processing, application of the liquid to one side of the film is sufficient, because, the active ingredient, once transported into the film, can form part of the complete film composition. Such method of application of an active ingredient can result in film products which have beneficial gradations in concentration of the active ingredient in the final film product. Also, such application may well inevitably result in a robust film containing active ingredient(s) and which is suitable for a variety of applications. Films produced by such a method are physically one single film, and may comprise 2 or more ‘bands’ or ‘areas’ in actual fact, such bands or areas having a degree of polymeric interaction with the film and or one another.

The association of the fluid, e.g. liquid with the building block film, and subsequent curing, as necessary, forms the product film.

In one aspect of the invention, the product film comprises a single homogenous polymer with the active ingredient evenly dispersed throughout the film, or in a concentration gradient within the film.

In another aspect of the invention, the product film comprises 2 or more bands associated with one another to a greater or lesser degree, with the active ingredient dispersed within particular band(s) only, or, to an extent, deposited on the surface (internal or external) of one or more bands in the product film.

It is to be understood that in this aspect of the invenyion the product film is comprises a single physical film. Such product film being robust for both storage and application, and which maintains sufficient integrity as such, for commercially viable use.

In this aspect of the invention, the product film does not comprise 2 or more discrete films simply adhered or bonded together.

The fluid and the building block film associate with one another to an extent where the association results in more than adhesion.

To described the product film in another way, the association of liquid and film results in a product film which, because of its own physical properties, cannot be physically split back into the original physical components from which it was formed i.e. the liquid (or cured result thereof) and the building block film under normal conditions, such films resulting from bands in the product film. Therefore, the product film always possesses a degree of structural homogeneity between at least 2 bands in the film, if those bands, indeed exist at all.

It is also to be understood that the association of the liquid with the building block film to form a product film results in a single film which may or may not have more than one band associated with it. These bands may comprise e.g., differences in polymer chemistry or polymer quality or differences in concentration levels of active ingredient. Indeed, these bands may consist of any differences or variations occurring within the film as a result of carrying out the process according to the present invention, in order to form the product film.

Appropriate means of liquid application onto the film substrate includes extrusion, roller application, pouring and leveling by doctor blade or knife, spraying, brush painting or wiping. As long as the surface application is uniform, the active ingredient is more easily evenly applied onto the film substrate. Preferably, but not essentially the liquid comprises at least one polymer which is compatible with the ‘building block’ film. The final ‘coated’ film composition may be conveniently left at ambient temperature and humidity in order to allow the assimulation of the ‘surface layer’, if applicable, to be transported into the body of the film substrate. To accelerate the process, the film substrate can be heated up to temperatures of 80° C. or the complete film transferred into a warm oven of similar temperature for a short period of time. A measure of when incorporation of active ingredient is complete is when the film surface becomes touch dry.

In many cases, where the liquid carrying the active ingredient, contains non-fugitive materials, a new film composition is produced. There may be instances where fugitive solvents are used in the liquid carrier but this would primarily be to accelerate the process of active ingredient transport.

As a further embodiment of the invention, two or more active ingredients or materials, may be selectively transported into the film substrate. In this instance, one active ingredient may have an affinity to move into the film substrate and the other may remain on the surface of the film, as a descreate band. An example of this would be a liquid formulation containing an active ingredient such as Ibuprofen and excipient such as sucrose. The two materials could be dissolved into a liquid formulation, which when applied as the transport medium to a film substrate, would result in the Ibuprofen moving into the core of the film and the sucrose, which has no affinity to be transported, remaining as a distinct surface layer on the film. This selectivity has a number of advantages, for example, the application of taste masking materials to a film surface.

Typical liquid materials which can be used to dissolve active ingredients or other compounds and act as selective transport mediums would primarily be polar liquids, which are predominantly water soluble or partially water soluble. These are mainly organic but inorganic materials such as water could also be used.

For a liquid to function as a suitable transport medium, compounds containing one or more of the following functional groups or compounds in its molecular structure, may be found suitable:
Hydroxy
Carboxy
Amino
Carboxamido
Epoxy
Oxo or keto
Cyano
Benzyl
Alkoxy or aryloxy
Furans, Pyrroles and thiophenes
Sulfoxide and sulfone
Quaternary nitrogen
Pyridine
Anhydrides
Esters and lactones

With a relatively broad range of polar organic liquid carriers to select from, it is possible to choose one or more compounds to act as a transport medium for a active ingredient. The active ingredient would ideally need to be soluble in the transport medium to work within this invention. Speed of active ingredient transport and end user application are controlled by the careful selection of polar liquids. For example fast transport of active ingredient into the film can be controlled by the molecular weight and functional groups within the liquid transport medium. Inevitably choice of liquid carrier is dictated by the end user application of the final film product, for example whether it can be safely ingested or, for topical applications, its acceptability for use on skin.

There is no limitation as to which type of medicament, drug, active ingredient, flavour, fragrance or agent which can be used in this final step of the process. Those skilled in the art of formulation science will choose a suitable polar liquid or formulation to work in conjunction with suitable cellulose film to achieve the desired application.

In another aspect of the present invention there is provided a method for producing a film incorporating active ingredients, wherein the one or more active ingredients are not exposed to the harsh conditions which may be necessary to produce a film e.g. the ‘building block’ film. A milder process, according to the present invention is then used to introduce the active ingredient to the film, advantageously avoiding subjecting the active ingredient to any unfavourable conditions in film production. This then facilitates the use of films which would otherwise be unsuitable for incorporating certain active ingredients, due to the initial conditions of film production. New useful film formulations are therefore contemplated.

The incorporation of active ingredients in a film in accordance with the invention also allows films to be produced which have active ingredients effectively applied and incorporated within the film in specific patterns. This also gives rise to the opportunity in further treating the film so that e.g. drugs can be release in a timed manner (e.g. parallel or sequential timing or both). For example, a film having a certain pattern of drug applied to it, can then be folded in a certain manner and fixed that way to produced a dosage form, such that certain drugs may be hidden deeper within the dosage form and are released later in time than those closer to the surface of the dosage form. In a similar way, films may be coiled, compressed and folded in a zig-zag manner or simply set in a multi-film ply formation, so form descrete calculated ‘thinking’ dosage forms, which are able to release actives in a complex manner, perhaps in accordance with timed release profile and/or differing external conditions.

Also contemplated are other dosage forms utilizing film or films made according to the present invention. For example a powder slug, tablet may be enrobed by such film or a liquid filled pharmaceutical capsule may be made for this film. It is easily contemplated e.g that there may be a situation wherein a liquid filled capsule contains a drug which is a stomach irritant but unfortunately needs to be release within the stomach. A film according to the present invention may be so designed as to incorporate a local anaesthetic. A capsule could thus be designed to release a drug (which is a stomach irritant) into the stomach, but which before such release, the capsule wall made from the film according to the present invention, itself releases a local anaesthetic to reduce the pain in the stomach.

Examples of the preparation and composition of film material containing medicament are as follows:

### EXAMPLE 1

Solution A)

| | | | |
|---|---|---|---|
| Hydroxypropyl methylcellulose* | 10.0% | 100 | g |
| Glycerin | 1.0% | 10 | g |
| Triethyl citrate | 1.0% | 10 | g |
| Purified water | 88% | 880 | g |

*Methocel E50 LV Premium (ex Dow chemicals)

The hydroxypropyl methylcellulose (HPMC) was carefully added to the purified water at 80° C. with stirring. This was followed by the addition of glycerin. The solution was cooled to 30° C., maintaining agitation to produce a colourless, viscous solution. Triethyl citrate was slowly added to the cooled solution with gentle mixing to produce a clear, viscous solution.

The solution was allowed to stand for 24 hours to allow it to naturally de-aerate. This resulting film forming solution was used to prepare films using an adjustable doctor blade (R.K. Print Coat Instruments Ltd, Royston, Herts). The gap on the doctor blade was set at 1.6 mm and used to draw down the solution onto a glass plate, which was then air dried for 24 hours at 25° C., 45% R.H.

Once dry this substrate film (film A) had evenly applied to its surface (surface which was in contact with the glass plate) a solution B of the following compositions:

Solution B)

| | | | |
|---|---|---|---|
| Ibuprofen | 13% | 13 | g |
| Propylene glycol | 56% | 56 | g |
| Triacetin | 27% | 27 | g |
| HPMC (Methocel E15 LV Premium) | 4% | 4 | g |

The HPMC was added to propylene glycol heated to 80° C. The mixture was shirred until a solution was formed then the triacetin was added and stirred to form a clear solution. This was cooled to 40° C. and then Ibuprofen was added. The mixture was shirred until all the Ibuprofen had dissolved resulting in a clear viscous solution. (Solution B). This solution, called the selective transport medium will be used in the next stage of the process.

Solution B) is evenly applied onto the surface of film A) using a doctor blade knife at a rate of 15 grams per sq metre of Solution B to 100 grams per sq metre of film A. This results in a total film mass of 115 g.s.m. The resulting film is left at room temperature for 30 minutes until the surface is touch dry and the selective transport medium (STM) and ibuprofen has reconstituted itself into the final film (film C).

The final composition of film C, which is a combination of film A and solution B is now:

| | % w/w |
|---|---|
| HPMC | 73.1 |
| Glycerin | 7.2 |
| Triethyl citrate | 7.2 |
| Propylene glycol | 7.3 |
| Triacetin | 3.5 |
| Ibuprofen | 1.7 |

The dry film can be converted into small units for use in oral administration.

Other examples of polar liquids mixtures which can be used to dissolve Ibuprofen and act as the selective transport medium are tabulated in table 1 (formulations 1-9). Each of the resulting solutions can be applied at the same rate as Solution B) in example 1 to the same film A, resulting in a different film composition in each case all contain the Ibuprofen as the medicament.

Table 2 demonstrates similar formulations (10-18) but in this case the medicament is a simple flavour.

Table 3 illustrates a range of formulations (19-23) based on ascorbic acid (vitamin C) as the medicament.

TABLE 1

| | Formula | | | | | | | | |
|---|---|---|---|---|---|---|---|---|---|
| Ingredient | 1 | 2 | 3 | 4 | 5 | 6 | 7 | 8 | 9 |
| Ibuprofen | 10.0 | 10.0 | 10.0 | 10.0 | 10.0 | 10.0 | 10.0 | 10.0 | 10.0 |
| Benzyl alcohol | 85.5 | | | | | | | | |
| Isobutanol | | 67.5 | | | | | | | |
| Propylene Glycol | | | 57.6 | | | | | | |
| 1,4-Butyrolactone | | | | 85.5 | | | | | |
| N-Methyl-2 Pyrrolidone | | | | | 85.5 | | | | |
| Ethanol | | | | | | 67.5 | | | |
| Triacetin | | | 27.9 | | | | | | |
| Monoacetin | | | | | | | 85.5 | | |
| Lactic acid | | | | | | | | 68.4 | |
| Glacial acetic acid | | | | | | | | | 58.5 |
| Water | | 18.0 | | | | 18.0 | | 17.1 | 27.0 |
| HPMC* | 4.5 | 4.5 | 4.5 | 4.5 | 4.5 | 4.5 | 4.5 | 4.5 | 4.5 |

*Methocel E50 LV Premium, ex Dow Chemicals (used as a viscosity aid)

TABLE 2

| | Formula | | | | | | | | |
|---|---|---|---|---|---|---|---|---|---|
| Ingredient | 10 | 11 | 12 | 13 | 14 | 15 | 16 | 17 | 18 |
| Cherry Flavour + | 20.0 | 20.0 | 20.0 | 20.0 | 20.0 | 20.0 | 20.0 | 20.0 | 20.0 |
| Benzyl alcohol | 76.0 | | | | | | | | |
| Isobutanol | | 60.0 | | | | | | | |
| Propylene Glycol | | | 51.2 | | | | | | |
| 1,4-Butyrolactone | | | | 76.0 | | | | | |
| N-Methyl-2 | | | | | 76.0 | | | | |
| Pyrrolidone | | | | | | | | | |
| Ethanol | | | | | | 60.0 | | | |
| Triacetin | | | 24.8 | | | | | | |
| Monoacetin | | | | | | | 76.0 | | |
| Lactic Acid | | | | | | | | 60.0 | |
| Glacial acetic acid | | | | | | | | | 52.0 |
| Water | | 16.0 | | | | 16.0 | | 16.0 | 24.0 |
| HPMC* | 4.0 | 4.0 | 4.0 | 4.0 | 4.0 | 4.0 | 4.0 | 4.0 | 4.0 |

+ Cherry flavour ex F. D. Copeland & Sons Ltd

TABLE 3

| | Formula | | | | | |
|---|---|---|---|---|---|---|
| Ingredient | 19 | 20 | 21 | 22 | 23 | 24 |
| Ascorbic Acid | 5.0 | 4.0 | 5.0 | 4.5 | 5.0 | 40.0 |
| Citric acid | 42.8 | | | | | |
| Lactic acid | | 60.0 | | | | |
| Propylene Glycol | | | 62.7 | | | |
| Benzyl alcohol | | | | 82.0 | | |
| Iso-butanol | | | | | 71.3 | |
| Water | 47.5 | 32.0 | 29.0 | 9.5 | 19.0 | 55.0 |
| HPMC* | 4.7 | 4.0 | 3.3 | 4.0 | 4.7 | 5.0 |

** Lactic acid provided in the form of a 90% solution, (Purac PH90 ™, ex Purac Biochem)

Each Formula (1 to 24) takes a finite time to incorporate and consolidate the medicament into a cellulose ether film substrate. Table 4 illustrates the length that each formula takes to constitute itself into a HPMC film.

The time value is based on each formula being applied at 25 gsm on a substrate HPMC film, 110 micros thick and around 150 gsm in weight. Applications of each liquid formulation were performed by means of a doctor blade. Conditioning environment was 21° C., 45% R.H. and the dry point was assessed when the applied surface became touch dry.

The resulting films were clear and free from particulate or crystalline matter.

TABLE 4

| Formula | Time Min |
|---|---|
| 1 | 3 |
| 2 | 1 |
| 3 | 5 |
| 4 | 5 |
| 5 | 2 |
| 6 | 2 |
| 7 | 10 |
| 8 | 17 |
| 9 | 3 |
| 10 | 2 |
| 11 | 1 |
| 12 | 8 |
| 13 | 2 |
| 14 | 3 |
| 15 | 2 |
| 16 | 15 |
| 17 | 10 |
| 18 | 3 |
| 19 | 2 |
| 20 | 2 |
| 21 | 6 |
| 22 | 3 |
| 23 | 2 |
| 24 | 3 |

Formulae 1 to 18 were applied to a film of the following composition:

| | % w/w |
|---|---|
| HPMC* | 80 |
| Glycerin | 10 |
| Triethyl citrate | 10 |

Formulae 19 to 24 were applied to a film of the following composition:

| | % w/w |
|---|---|
| HPMC* | 77 |
| Glycerin | 3 |
| Citric acid | 20 |

(Table 5) illustrates examples of formulations which behave as selective transport mediums. All contain active ingredients which can be incorporated into HPMC film

*Methocel E50 LV Premium, Ex Dow Chemicals

TABLE 5

| | Formula | | | | | |
|---|---|---|---|---|---|---|
| Ingredient | 25 | 26 | 27 | 28 | 29 | 30 |
| Ibuprofen | 25 | | | | | |
| Ascorbic Acid | | 40.0 | | | | |
| Menthol | | | 50.0 | | | |
| Dextromethorphan HBr | | | | 10.0 | | |
| Alpha Tocopherol | | | | | 50.0 | 50.0 |
| Propylene Glycol | 75 | | | 90 | | |
| Ethanol | | | | | 50.0 | |
| Benzyl alcohol | | | 50.0 | | | 50.0 |
| Water | | 58.0 | | | | |
| HPMC | | 2.0 | | | | |
| Dry Point (Mins) | 7 | 5 | 10 | 7 | 2 | 4 |

Each formula (25 to 30) was applied to the surface of a substrate HPMC film at a rate of 25 g.s.m. the substrate film was 115 microns thick and around 160 g.s.m. in weight. Application was performed by means of a doctor blade and the liquid formulation was allowed to consolidate itself into the HPMC film substrate. Conditioning environment was 21° C., 45% RH. The dry point was recorded in Table 5.

Film substrate composition was:

| | |
|---|---|
| HPMC (E50) | 77% |
| Glycerin | 3% |
| Citric acid | 20% |

The final films remain clear and free from particulate or crystalline matter.

### DESCRIPTION OF THE DRAWINGS

The following drawings are intended to further describe the invention, by way of example, and are not intended to limit the invention in any way.

FIG. 1 shows how the liquid can be applied to the film. Liquid solution containing medicament (1) (which may form the transport medium) is introduced to the film substrate (2). Application of liquid solution containing medicament on film substrate (3), controlled by doctor blade (4) is shown and resulting association of medicament in film (5) to form the final product film (6).

FIGS. 2-4 show the various stages in the assimilation of e.g. active ingredients. FIG. 2 shows the film substrate (3) without the active ingredient and FIG. 3 shows a liquid solution containing a medicament (2) (transport medium) resting on the surface of a film at time=0.

FIG. 4 shows, with the passage of time, the film product (6) with the association of medicament in film (5) at time=X.

FIG. 5 shows an arrangement in which the product film may be folded to form a dosage form. Liquid solutions containing medicaments (a+b) can be applied to both sides of the film at time=0. This film system can be folded into a compressed zig-zag, as shown in FIG. 6, where compressed sections of the film product may be fused or laminated together to form a solid composite with the medicament striated or otherwise distributed within the product.

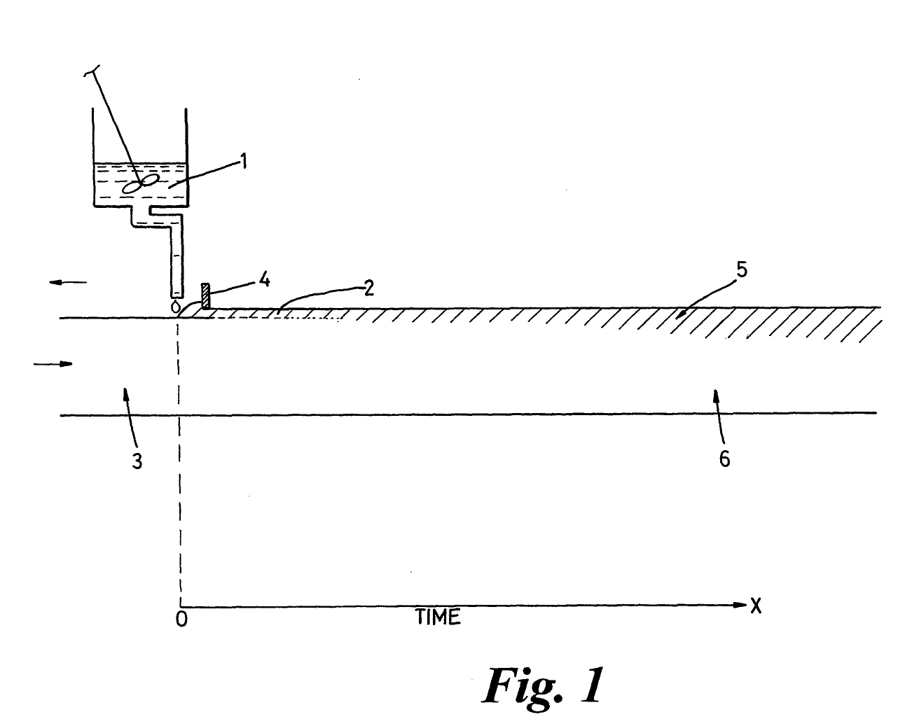

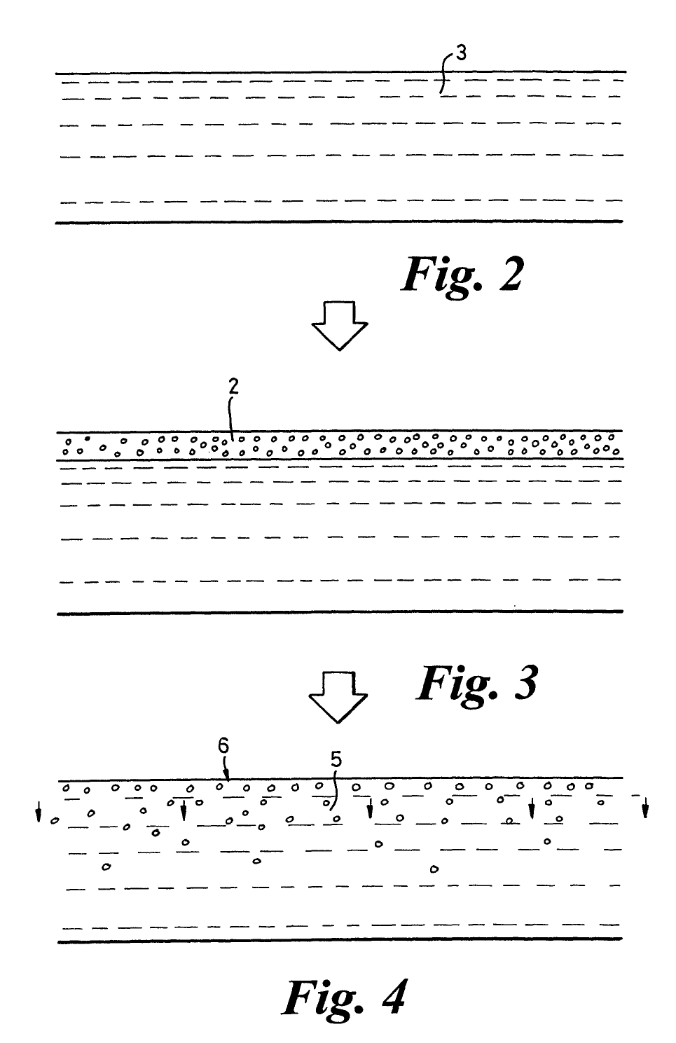

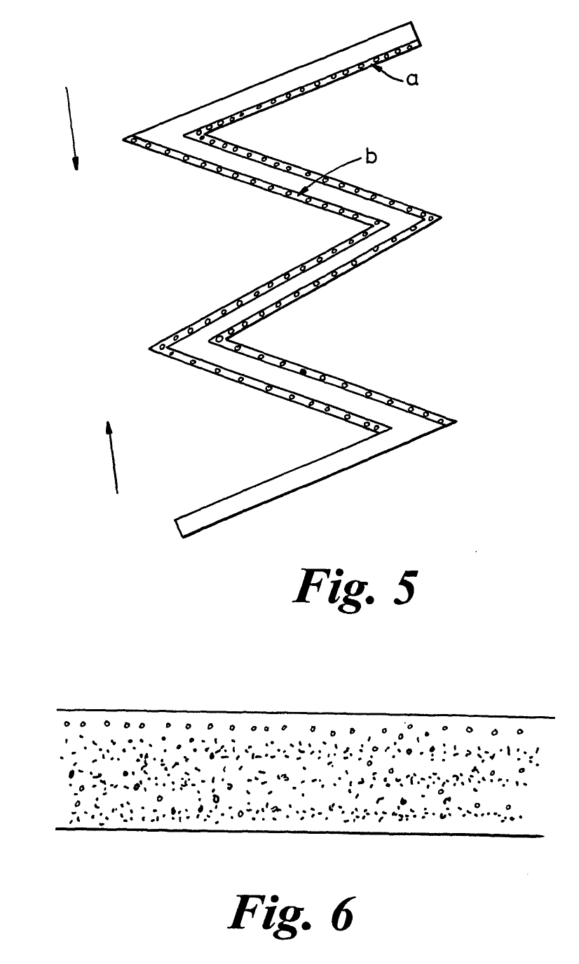

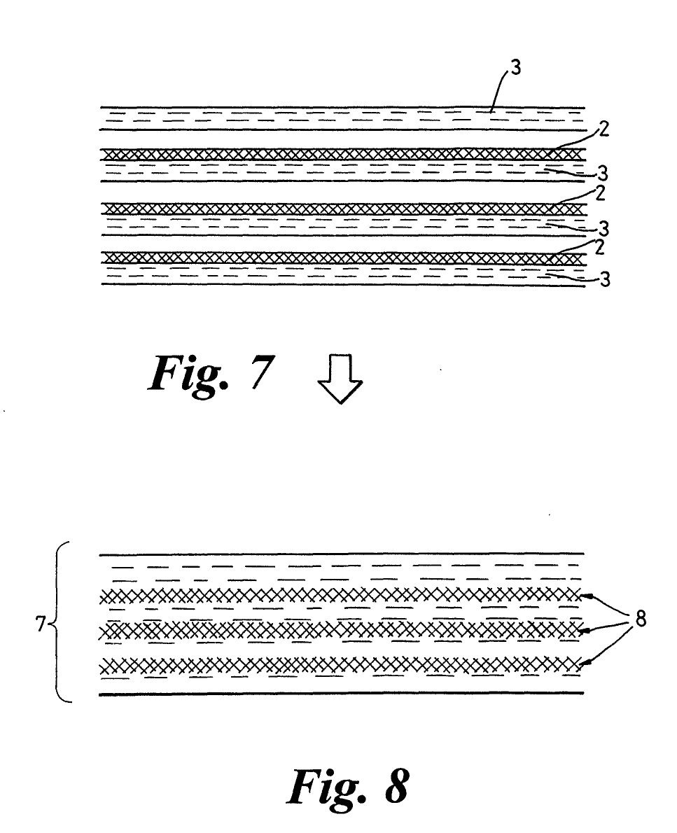

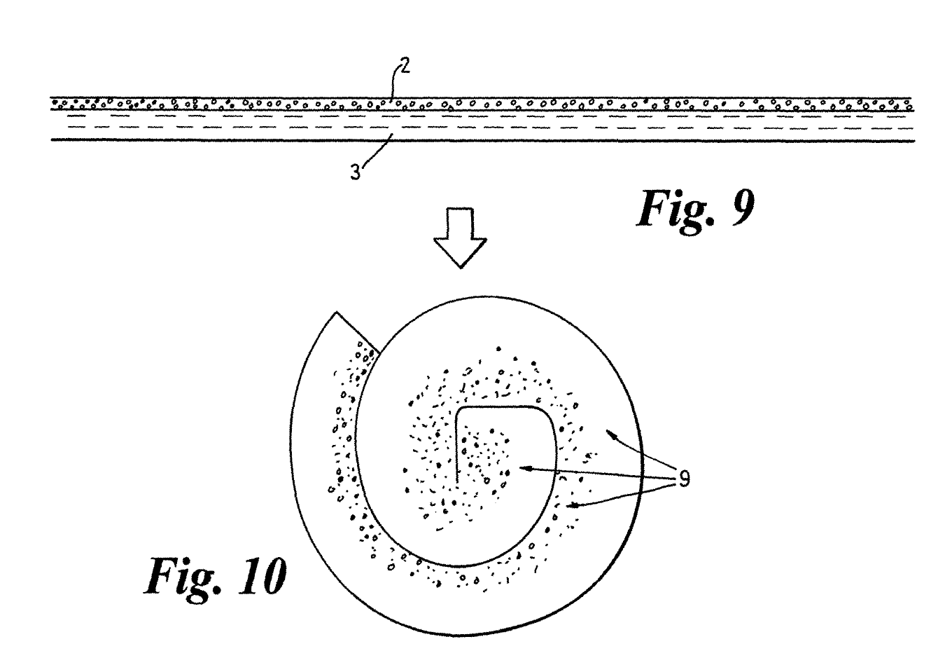

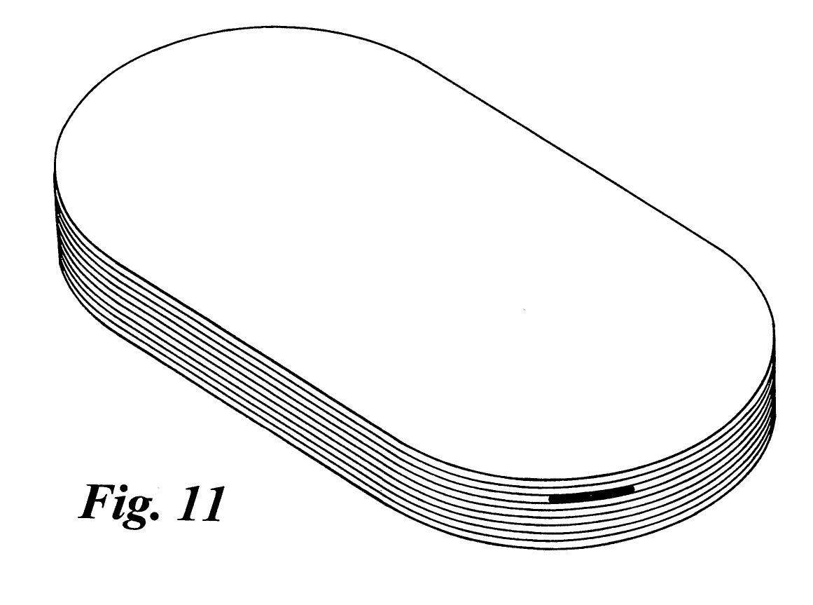

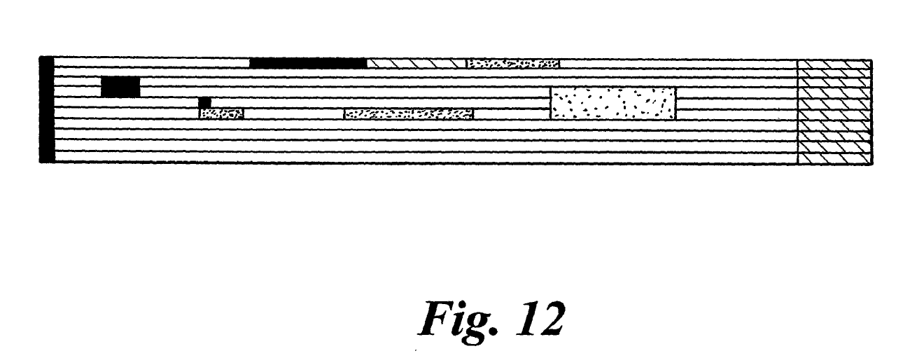

1. A method of producing a single, discrete non-gelatin film comprising:
a) forming a non-gelatin polymeric film, with or without active ingredients incorporated therein;
b) applying a liquid to one or more surfaces of the film, said liquid incorporating at least one active ingredient; and
c) allowing the liquid applied to at least partially cure and associate with the film, to result in;
the fluid being absorbed within the film, wholly or partially, and forming a homogeneous polymer film product.

2. A method according to claim 1, wherein the non-gelatin film produced comprises one or more layers which associate with one another to a lesser or greater degree to form a partially or wholly polymerically homogeneous film.

3. A method according to claim 1, wherein the polymeric mass of the film or films is increased marginally or substantially after steps b) or c).

4. (canceled)

5. A method according to claim 1 whereby one or more polymeric substances are also deposited on the film surface.

6. A method according to claim 1, wherein the active ingredient in the liquid is transported onto or into the film during step c) of claim 1.

7. A method according to claim 2 wherein the active ingredient is selectively transported.

8. A method according to claim 1, wherein the non-gelatin film comprises a cellulose ether film.

9. A method according to claim 1, wherein the non-gelatin film comprises one or more of the following polymers:
hydroxypropyl methylcellulose (HPMC),
hydroxy propyl cellulose (HPC),
hydroxy ethyl methyl cellulose (HEMC),
hydroxy ethyl cellulose (HEC),
methyl cellulose (MC),
carboxy methylcellulose (CMC),
sodium carboxy methylcellulose
and salts and derivatives of all aforesaid.

10. A method according to claim 1, wherein the liquid comprises a same or similar polymeric material as to which forms the non-gelatin film.

11. A method according to claim 1, wherein the liquid comprises a material which is chemically or physically compatible with the material which forms the non-gelatin film.

12. A method according to claim 1, wherein the active ingredient is transported from the liquid to the film.

13. A method according to claim 1, wherein the active ingredient has a higher affinity for the liquid than the film.

14. A method according to claim 1, wherein the film active ingredient has a higher affinity for the film than the liquid.

15. A method according to claim 10, wherein 2 or more active ingredients have the same or differing affinities for the film and liquid.

16. A film produced by the method in claim 1.

17. (canceled)

18. A film according to claim 16, wherein one or more active ingredients are present in the film and which have concentration gradients associated with one or more bands or patterns within the film.

19. A film produced according to claim 1, wherein the active ingredient continues to move or be transported after the curing stage.

20. A film according to claim 16, wherein one or more layers associate with one another to a lesser or greater degree to form a level of polymeric homogeneity.

21. A film according to claim 16, which is coiled.

22. A film according to claim 16, which is folded in a zig-zag formation.

23. A pharmaceutical dosage form comprising multi-layers of film formed from films according to claim 16.

24. A pharmaceutical dosage form according to claim 23, wherein the films are laid together before any liquid or transport medium applied has cured or dried.

25. A film produced in accordance with claim 1, wherein the film is packaged to form a dose unit.

26. A sheet of film produced in accordance with claim 1, wherein the film has liquid according to claim 1 applied to it, on one or both sides, and on opposing/adjacent areas or non-opposing or adjacent areas or overlapping areas to form a pattern.

27. A dosage form produced from a sheet of film wherein the pattern produced with respect to the film provides a delayed active drug release profile.

28. A pharmaceutical dosage form derived from a film produced by a method according to claim 1.

29. Use of a film according to claim 16, wherein the film is placed on the tongue of a human or animal and the active ingredients are released in a convenient manner as the film disintegrates.

30. A tablet, powder slug or capsule made from or coated, enrobed or encapsulated with a film according to claim 16.

31. A tablet or monolith made from multiple layers of film according to claim 16.

32. A tablet or monolith according to claim 25, wherein said tablet or monolith comprises three to forty layers.

33. A tablet or monolith according to claim 25, wherein said tablet or monolith comprises 8 to 25 layers.

34. A tablet of monolith according to claim 25, wherein the tablet or monolith comprises 10 to 20 layers.

35. A multicellular dosage form made from a film according to claim 16.

36. A method according to claim 10, wherein said liquid comprises a material which is chemically or physically compatible with the material which forms the non-gelatin film, and wherein 2 or more active ingredients have the same or differing affinities for the film and liquid.

37. A film according to claim 18, which is coiled.

38. A film according to claim 19, which is coiled.

39. A film according to claim 20, which is coiled.
